(19) United States
(12) Patent Application Publication
(10) Pub. No.: US 2025/0152085 A1
(43) Pub. Date: May 15, 2025

(54) GASTRIC ACTIVITY ANALYSIS SYSTEM / DEVICE / METHOD

(71) Applicant: Alimetry Limited, Auckland (NZ)

(72) Inventors: Gabriel SCHAMBERG, Auckland (NZ); Armen Alex GHARIBANS, Auckland (NZ); Gregory Brian O’GRADY, Auckland (NZ); Chris VARGHESE, Auckland (NZ); Stefan Sam Scott CALDER, Auckland (NZ)

(21) Appl. No.: 18/389,450

(22) Filed: Nov. 14, 2023

(51) Int. Cl. A61B 5/00; A61B 5/053; G16H 40/67

(52) U.S. Cl. CPC A61B 5/4238; A61B 5/053; A61B 5/4842; A61B 5/6833; G16H 40/67; A61B 5/742

(57) ABSTRACT

A method including monitoring a patient's gastric activity by obtaining data based on spectral gastric activity with an electrode array over a predetermined test period, concurrently obtaining patient symptom information for a predetermined set of symptoms during at least a portion of the test period, determining a normalized gastric activity amplitude over at least a portion of the test period from the measured gastric activity data, and correlating over at least a portion of the test period said patient symptom information with said normalized gastric activity amplitude and determining a measure of said correlation and at least one of: treating for a gut-brain axis disorder if the measure indicates a correlation is absent, optionally by not satisfying a predetermined correlation threshold or treating for gastric dysfunction if the measure of said correlation indicates a correlation exists, optionally by satisfying a predetermined correlation threshold.

Embodiments include teachings associated by way of example only with an electrode patch and a gastric activity detection system and/or device and/or methods associated therewith.

Embodiments include a gastric activity detection system and/or device and/or methods suitable for use in monitoring gastro-intestinal electrical activity, and human patient care.

### BACKGROUND

Chronic gastro-duodenal symptoms affect more than 10% of the global population and have a significant economic impact, resulting in a significant healthcare burden.

### SUMMARY

Embodiments can provide a gastric activity detection system and/or symptom profiling system and/or phenotyping system (and/or related device and/or methods associated therewith) which provides utilitarian value and alleviates risks of prior implementations and also can, in some instances, provide alternatives to prior implementation of systems focusing on gastric activity evaluation.

Some embodiments are directed to monitoring/analyzing/detecting gastric symptoms in human patients. Embodiments provide devices/systems and/or methods for/of identifying and/or differentiating between various specific mechanisms contributing to an individual patient's symptoms. In some embodiments, the teachings herein are directed to identifying abnormal gastric motility in a significant subgroup of patients with chronic gastric conditions. In some embodiments, the teachings herein are directed to identifying gastric motility that affects only a subset of people falling within a group in the overall population that are less than, greater than and/or equal to 1, 2, 3, 4, 5, 6, 7, 8, 9, 10, 11, 12, 13, 14, 15, 16, 17, 18, 19 or 20% or any value or range of values therebetween in 1% increments. Embodiments provide a reliable method for assessing gastric motor function in clinical practice. Embodiments can provide correct assessment at at least a 75, 80, 85, 90, 91, 92, 93, 94, 95, 96, 97, 98 or 99% or greater rate or any value or range of values therebetween in 1% increments out of at least 50, 100, 200, 300, 400 or 500 or more random patients.

Embodiments include utilizing patient reported symptoms as a useful foundation for clinical assessment and diagnosis of gastro-duodenal disorders. Embodiments provide superior results over the Rome diagnostic questionnaire. Embodiments can avoid the classification of the presence or absence of symptoms over a long timeframe (typically months), and can avoid significant overlap and/or can provide utilitarian insight into symptom aetiology.

Embodiments can include continuous or semi/continuous assessment of symptom severity particularly after a meal stimulus for the purposes of diagnostic data collection.

Embodiments can provide a standardised system for quantitative assessment of an individual patient. Embodiments can include Body Surface Gastric Mapping (BSGM) that employs multi-electrode arrays to measure and map gastric myoelectrical activity. In particular, BSGM can be used in some embodiments to provide high-quality and high resolution information noninvasively.

Embodiments can include least semi-automated digital and/or analogue tools developed for obtaining standardised gastric symptom profiling, simultaneously during testing in order to aid in the identification of specific disease phenotypes.

In an exemplary embodiment the invention comprises a method, comprising:
monitoring a patient's gastric activity by:
obtaining data based on spectral gastric activity with an electrode array over a predetermined test period;
concurrently obtaining patient symptom information for a predetermined set of symptoms during at least a portion of the test period;
determining a normalized gastric activity amplitude over at least a portion of the test period from the measured gastric activity data; and
correlating over at least a portion of the test period said patient symptom information with said normalized gastric activity amplitude;
determining a measure of said correlation; and at least one of:
   (i) treating for a gut-brain axis disorder if the measure indicates a correlation is absent, optionally by not satisfying a predetermined correlation threshold; or
   (ii) treating for gastric dysfunction if the measure of said correlation indicates a correlation exists, optionally by satisfying a predetermined correlation threshold.

According to another aspect the method further comprises the patient ingesting a predetermined standardized meal preferably at a predetermined time during the test period.

According to another aspect the invention comprises a method, comprising:
obtaining data based on measured spectral gastric activity measured with an electrode array during a first temporal test period;
obtaining data based on patient symptom information for a predetermined set of symptoms, wherein the patient symptom information was obtained during at least a portion of the first temporal test period;
determining data indicative of normalized gastric activity amplitude from the obtained data based on the measured gastric activity data;
correlating said patient symptom information with said data indicative of normalized gastric activity amplitude over the test period;
evaluating the correlation; and identifying a treatment for a gut-brain axis disorder if the evaluation of the correlation indicates no clinical correlation exists.

According to another aspect the invention comprises a method, comprising:
obtaining first data based on measured spectral gastric activity measured with an electrode array during a first temporal test period;
obtaining second data based on patient symptom information for a predetermined set of symptoms, wherein the patient symptom information was obtained during at least a portion of the first temporal test period;
providing the first and second data;
receiving third data; and
prescribing a treatment based on the third data, wherein
the third data is an evaluation of a correlation of the first data with data indicative of normalized gastric activity amplitude from the second data.

According to another aspect said patient symptom information is obtained based on output from a patient who assigned a symptom severity metric at intervals during the test period.

According to another aspect said measure of said correlation is displayed or output in a digital or physical medium.

According to another aspect said symptom information is obtained for said set of symptoms based on one or more of, or an average of two or more of:
a) nausea
b) bloating
c) upper gut pain
d) heartburn
e) stomach burn, and
f) excessive fullness.

According to another aspect said measuring of gastric activity with an electrode array further includes spatial information of said gastric activity.

According to another aspect said treating for a gut-brain axis disorder only occurs when said spectral gastric activity is indicated as normal.

According to another aspect the invention comprises a method, comprising:
monitoring a patient's gastric activity by:
measuring spectral gastric activity with an electrode patch over a predetermined test period;
concurrently obtaining patient symptom information over at least a portion of the test period;
determining a measure of gastric activity amplitude from the measured gastric activity data; and
correlating said patient symptom information with said gastric activity amplitude over the at least a portion of the test period;
determining a measure of said correlation, and classifying a patient into one of a predetermined phenotype set based on the determined measure of said correlation.

According to another aspect the method further comprises the patient ingesting a predetermined standardized meal, preferably at a predetermined time during the test period.

According to another aspect said phenotype set is one or more of:
a) Sensorimotor, if indicative of synchronisation between symptom severity and gastric amplitude
b) Post-gastric, if indicative of symptom severity following gastric activity
c) Activity alleviated, if indicative of symptoms preceding gastric activity
d) Continuous, if indicative of symptom severity being high and optionally consistent throughout the test and/or
   a lack of correlation between gastric amplitude and symptom severity, where symptom severity is high and optionally consistent throughout the test.

According to another aspect said measuring of gastric activity with an electrode array, further includes spatial information of said gastric activity.

According to another aspect the portion of the predetermined test period is at least approximately 45 minutes.

According to another aspect the invention comprises a system comprising:
electronics including a data input and a data output; and
an electrode assembly including a plurality of electrodes, the electrode assembly being configured to be placed on an abdomen of a human and maintained thereon for at least a predetermined test period, wherein
the electrode assembly is in signal communication with the data input,
the system includes a second data input configured to receive data related to severity, based on assessment by the human, of a predetermined set of symptoms;
the electronics is configured output information based on signals received from the electrode assembly via the data input and the second data input in a temporally correlated and/or correlatable manner, the system executing the method of any one or more of the clauses herein.

According to another aspect the invention comprises a system comprising:
an input suite configured to receive data based on data recorded from electrodes on an abdomen of a human and receive data based on a state of human perceptions of predetermined symptoms;
memory configured to store {if only in a transitory manner} the received data; and
electronics configured to automatically evaluate the received data and indicate a whether the human suffers from one or more predetermined gastric aliments based on the evaluation, the system executing the method of any one or more of the clauses herein.

### Sensorimotor

According to another aspect the invention comprises a method comprising monitoring a patient's gastric activity by:
obtaining data based on measured spectral gastric activity over a predetermined test period;
obtaining patient symptom information based on data obtained from the patient during at least a portion of the test period, and determining a normalized continuous symptom severity function;
determining normalized gastric activity amplitude over at least a portion of the test period based on data the measured gastric activity data; and
determining a degree of temporal association between the normalised gastric amplitude and normalised continuous symptom severity functions for a symptom selected from a predetermined set of symptoms and/or for an average symptom function for two or more symptoms selected from the predetermined set of symptoms.

According to another aspect the method further comprises the patient ingesting a predetermined standardized meal preferably at a predetermined time during the test period.

According to another aspect said predetermined set of symptoms are chosen from one or more of, or an average of two or more of:
a) nausea
b) bloating
c) upper gut pain
d) heartburn
e) stomach burn, and
f) excessive fullness.

According to another aspect the invention further comprising classifying the patient for example as Sensorimotor phenotype when synchronization between normalised gastric amplitude and normalised symptom severity functions is identified.

According to another aspect the invention further comprising calculating a temporal correlation coefficient and assessing based on the coefficient the temporal synchronization of the normalised gastric activity amplitude and a normalised symptom severity curve.

According to another aspect the temporal correlation coefficient is calculated for each symptom severity curve if a standard deviation is above a predetermined standard deviation threshold.

According to another aspect the temporal correlation coefficient is calculated for the average normalized symptom severity curve if a standard deviation is above a predetermined deviation threshold.

According to another aspect the invention further comprising calculating an amplitude correlation coefficient if a standard deviation of the gastric amplitude function is above a predetermined gastric amplitude deviation threshold.

According to another aspect wherein the temporal correlation coefficient (for example Pearson's r) is calculated between the normalised gastric amplitude function and normalized symptom severity function for lags of ranging from approximately −10 to +10 minutes, with approximately 1 minute steps, and the correlation is taken as the maximum of these values.

According to another aspect a maximum temporal correlation coefficient is used to determine a phenotype for temporal associations between normalised gastric amplitude and normalised symptom severity.

According to another aspect a Sensorimotor phenotype is indicated when the maximum temporal correlation coefficient is greater than 0.5.

According to another aspect said measuring of spectral gastric activity with an electrode array further includes spatial information of said gastric activity.

According to another aspect the method further includes measuring spatial information of the gastric activity and the identification of the treatment and/or ailment and/or symptom is also based on the measured spatial information.

According to another aspect a determined Sensorimotor phenotype is used by a clinician to provide targeted therapies to a patient by treating as postprandial distress syndrome,

### Post Gastric

According to another aspect the invention comprises a method comprising monitoring a patient's gastric activity by:
measuring spectral gastric activity with an electrode patch over a predetermined test period;
concurrently obtaining patient symptom information over at least a portion of the test period for one or more predetermined symptoms, and determining a normalized continuous symptom severity function,
determining a normalized gastric activity amplitude function from the measured gastric activity data; and
identifying a time lag between the gastric amplitude and one or more symptom severity functions.

According to another aspect the method further comprises the patient ingesting a predetermined standardized meal preferably at a predetermined time during the test period.

According to another aspect an average difference between cumulative distribution functions (CDFs) is used to assess the time lag between normalised gastric amplitude and a normalised continuous symptom severity function.

According to another aspect the correlation coefficient is calculated for one or more respective symptom severity curves if a standard deviation is above a predetermined deviation threshold.

According to another aspect the predetermined deviation threshold is 0.5.

According to another aspect the correlation coefficient is calculated for the average symptom severity function if a standard deviation is above a predetermined deviation threshold.

According to another aspect the predetermined deviation threshold is 0.1.

According to another aspect the gastric amplitude function is normalized by:
a. The minimum value of the gastric amplitude function being subtracted from the whole function, and
b. The gastric amplitude function is divided by its sum.

According to another aspect the time lag is quantified as the average difference between the CDF of the normalised gastric amplitude function and the CDF of the normalised symptom severity function.

According to another aspect the time lag is thresholded to determine phenotypes associated with symptoms that either precede or follow gastric activity according to:
a. Post-Gastric phenotype of symptoms following gastric activity is indicated if said time lag is greater than 0.25, or
b. Activity-Alleviated phenotype if symptoms preceding gastric activity is indicated when said time lag is less than −0.25.

According to another aspect said measuring of spectral gastric activity with an electrode array, further includes spatial information of said gastric activity.

According to another aspect the determined phenotypes are used by a clinician to provide targeted therapies to patients according to:
a) The Post-Gastric phenotype may be treated as having small bowel/biliary causes,
b) The Activity-Alleviated phenotype may be treated using a neuromodulator such as mirtazapine,

### Continuous

According to another aspect the invention comprises a method comprising monitoring a patient's gastric activity by:
measuring spectral gastric activity with an electrode patch over a predetermined test period;
concurrently obtaining patient symptom information over at least a portion of the test period, and determining a normalized continuous symptom severity function,
determining a normalized gastric activity amplitude function from the measured gastric activity data; and
identifying a lack of temporal correlation, that is determining that the gastric amplitude function does not follow or precede the normalised symptom severity function, and determining a symptom severity score from said continuous symptom severity function.

According to another aspect the method further comprises the patient ingesting a predetermined standardized meal preferably at a predetermined time during the test period.

According to another aspect the patient is classified for example as Continuous phenotype when said lack of temporal correlation is identified and said symptom severity score indicates the patient's symptom burden is high and consistent throughout the test period.

According to another aspect said measuring of spectral gastric activity with an electrode array, further includes spatial information of said gastric activity.

According to another aspect the invention comprises a method, comprising:
monitoring a patient's gastric activity by:
measuring spectral gastric activity with an electrode array over a predetermined test period;
concurrently obtaining patient symptom information for a predetermined set of symptoms over at least a portion of the test period;
determining a measure of gastric activity amplitude from the measured gastric activity data; and
   displaying a normalised gastric amplitude curve and at least one normalised symptom severity curve over the at least a portion of the test period, overlaid on the same set of axes, and
   determining a measure of correlation visually, and classifying a patient into one of a predetermined phenotype set based on the visually determined measure of said correlation.

According to another aspect the method further comprises the patient ingesting a predetermined standardized meal preferably at a predetermined time during the test period.

According to another aspect the gastric amplitude and symptom severity curves are normalized by:
a) by subtracting the mean and dividing by the standard deviation of each curve, and
b) The normalization is only performed when the curves have a standard deviation larger than zero.

According to another aspect the time of the meal is indicated on the display overlaid on the same set of axes.

According to another aspect the determined phenotypes are used by a clinician to provide targeted therapies to patients according to:
a) The Sensorimotor phenotype may be treated as postprandial distress syndrome,
b) The Post-Gastric phenotype may be treated as having small bowel/biliary causes,
c) The Activity-Alleviated phenotype may be treated using a neuromodulator such as mirtazapine,
d) The Continuous phenotype may be treated as epigastric pain syndrome or a disorder of gut-brain interaction.

Other embodiments and variations thereof will become apparent from the following description which is given by way of example only and with reference to the accompanying drawings.

In this specification where reference has been made to patent specifications, other external documents, or other sources of information, this is generally for the purpose of providing a context for discussing the features of the teachings herein. Unless specifically stated otherwise, reference to such external documents is not to be construed as an admission that such documents, or such sources of information, in any jurisdiction, are prior art, or form part of the common general knowledge in the art.

For purposes of the description hereinafter, the terms “upper”, “lower”, “right”, “left”, “vertical”, “horizontal”, “top”, “bottom”, “lateral”, “longitudinal” and derivatives thereof shall relate to the teachings herein as it is oriented in the drawing figures. However, it is to be understood that the variations of the teachings herein may assume various alternative variations, except where expressly specified to the contrary. It is also to be understood that the specific devices illustrated in the attached drawings and described in the following description are simply exemplary embodiments. Hence, specific dimensions and other physical characteristics related to the embodiments disclosed herein are not to be considered as limiting.

It is acknowledged that the term “comprise” may, under varying jurisdictions, be attributed with either an exclusive or an inclusive meaning. For the purpose of this specification, and unless otherwise noted, the term ‘comprise’ shall have an inclusive meaning, allowing for inclusion of not only the listed components or elements, but also other non-specified components or elements. The terms ‘comprises’ or ‘comprised’ or ‘comprising’ have a similar meaning when used in relation to the system or to one or more steps in a method or process.

As used hereinbefore and hereinafter, the term “and/or” means “and” or “or”, or both.

As used hereinbefore and hereinafter, “(s)” following a noun means the plural and/or singular forms of the noun.

When used in the claims and unless stated otherwise, the word ‘for’ is to be interpreted to mean only ‘suitable for’, and not for example, specifically ‘adapted’ or ‘configured’ for the purpose that is stated.

As used hereinbefore and hereinafter, the term “continuous” or “semi continuous” with respect to the test period is to be interpreted as ongoing throughout the entire or nearly entire test period.

### BRIEF DESCRIPTION OF THE DRAWINGS

Some embodiments of the invention will be described by way of example only and with reference to the drawings, in which:

FIG. 1 is an image of a body surface gastric mapping system shown on a patient wearing a sensing electrode array and data acquisition device.

FIG. 2 is a detailed plan view of the sensing electrode array of FIG. 1.

FIG. 3 is pictographic representation of steps to set up the gastric activity reader system. Part A details basic steps to prepare array on a patient. Part B illustrates coupling of array with reader/data acquisition device, and electrode signal check. Part C illustrates an example App used by a patient for example, to log real time (and concurrent) symptom data and an example report of patient symptom data (i.e. the symptom severity curve) over the time of the test.

FIG. 4 is a graphic representation of the body surface gastric mapping system. Part A shows example process steps for analysing raw electrode signal data, into Spectral analysis and/or Spatial analysis. Part B shows example spectral visual outputs from raw data, and example spatial visual outputs from raw data. Part C is a typical gastric activity spectral output graph showing calculated properties such as Rhythm Index, Principal Gastric Frequency, Fed:Fasted Amplitude ratio, and average amplitude.

FIG. 5 is an output plot for a typical (normal) patient, showing a spectral plot, a plot of amplitude extracted from the raw electrode data, and a plot of overall symptom burden extracted from the symptom logging App.

FIG. 6 is an output plot for a typical “Sensorimotor” patient, showing a spectral plot, a plot of amplitude extracted from the raw electrode data, and a plot of overall symptom burden extracted from the symptom logging App.

FIG. 7 is an output plot for a typical “Activity—Relieved” patient, showing a spectral plot, a plot of amplitude extracted from the raw electrode data, and a plot of overall symptom burden extracted from the symptom logging App.

FIG. 8 is an output plot for a typical “Continuous” patient, showing a spectral plot, a plot of amplitude extracted from the raw electrode data, and a plot of overall symptom burden extracted from the symptom logging App.

FIG. 9 is an output plot for a typical “Post-Gastric” patient, showing a spectral plot, a plot of amplitude extracted from the raw electrode data, and a plot of overall symptom burden extracted from the symptom logging App.

### DETAILED DESCRIPTION OF SOME EMBODIMENTS

Embodiments of the teachings herein can allow for the gathering, combination and analysis of multiple data sources potentially relevant to understanding gastric dysfunction. In particular, gastric activity data is measured (particularly with respect to post meal stimulus), while simultaneously/contemporaneously gathering temporally synchronized patient symptom information across a test period.

### Gastric Activity

Medical apparatus for monitoring electrical activity may use a sensing device comprising an electrode patch or a plurality of patches having one or more electrodes and a connector device (or devices) which could be an electronic device such as a data acquisition device that is in electronic communication with such patch. Also, it is utilitarian to provide an electrode patch connection system for a non-invasive medical apparatus that can be worn by a subject to monitor the physiological condition in a comfortable and reliable manner, while the subject is engaged in normal daily activities, and/or in a clinical test setting.

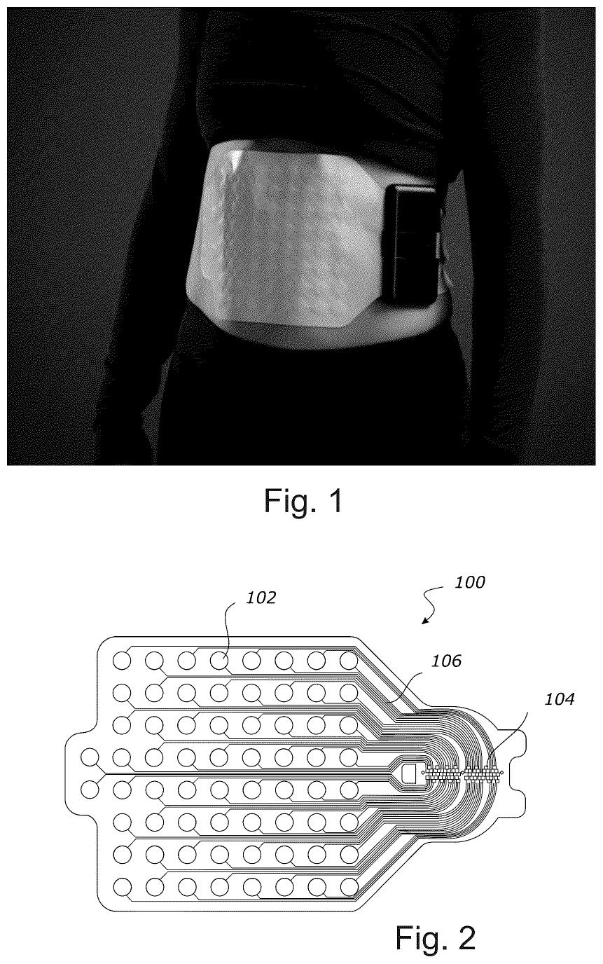

FIGS. 1 and 2 shows an exemplary electrode patch 100 that is configured to be used as part of a system for monitoring physiological functions on a subject. The subject is a human in some implementations but optionally the subject may be a non-human animal. The electrode patch 100 is configured in some embodiments to be used as part of a system for monitoring gastro-intestinal (GI) electrical activity of a subject. In some embodiments, the electrode patch 100 may be configured to monitor electrical/physiological activity on other regions of the subject such as but not limited to colonic regions.

The electrode patch 100 is a sensing device and comprises a plurality of spatially arranged surface electrophysiological sensors in the form of electrodes 102 for contacting an outer surface of the skin of the subject to sense and measure electrical potentials at multiple electrodes.

In the example shown in FIG. 2, there are total of 66 electrodes out of which 64 electrodes are arranged in an array of 8 rows and 8 columns and the remain two electrodes are the ground and reference electrodes, all by way of example. In use, electrical potentials may be measured as the difference between each of the 64 electrodes and the reference electrode. The ground electrode may be the “driven right leg” or “bias” electrode. The purpose of the ground electrode in some embodiments is to keep voltage level of the subject's body within an acceptable range and to minimize any common-mode in the subject's body (e.g., 50/60 Hz power-line noise). The driven right leg will act as a source or sink as utilitarian (within reason) to accomplish this. However, the electrode patch 100 may comprise more than 66 electrodes or less than 66 electrodes. The ground and reference electrodes may be different than what is shown in FIG. 2. In an embodiment, the patch (or the total system comprising less than, greater than and/or equal to 2, 3, 4, 5, 6, 7, 8, 9, 10, 11, 12, 13, 14, 15, 16, 17, 18, 19, 20 or more patches or any value or range of values therebetween in 1 increment) can comprise less than, greater than and/or equal to 10, 15, 20, 25, 30, 35, 40, 45, 50, 55, 60, 65, 70, 75, 80, 85, 90, 95, 100, 125, 150, 175, 200, 225, 250, 275 or 300 more or any value or range of values there between in 1 increment (e.g., 33, 94, 44 to 192, etc.).

In some embodiments, the electrode patch 100 is configured to be removably attached to the outer surface of the skin of the subject, such as for example at or near an abdominal region (as shown in FIG. 1), so that the electrodes 102, (a sufficient/utilitarian number, which could be a subset of the total number of channels—in an embodiment), can contact the outer surface of the skin of the subject at or near the abdominal region to sense and measure electrical signals from the GI tract of the subject. If the electrode patch 100 is for sensing and measuring electrical signals from other regions, then the electrode patch may be configured to be removably attached to the outer surface of the skin of the subject at or near at suitable regions, so that the electrodes 102, can contact the outer surface of the skin of the subject at or near such region to sense and measure electrical signals from the that region of subject's body. This may include a colonic region.

The electrode patch 100 and data acquisition system can be, by way of example only and not by way of limitation, as described in International Patent Application Publication No. WO2021/130683. In this example system electrical traces 106 are provided to connect each electrode 102, to a respective contact pad 104, for operatively coupling with a data acquisition device. The system may comprise a docking device having a compartment that is configured to receive the data acquisition device of the sensor array. The docking device may be a wireless charging device for facilitating wireless charging of the data acquisition device when docked.

The electrode patch and data acquisition system allows body surface gastric mapping (BSGM) information to be obtained in an autonomous, or at least semi-autonomous manner. BSGM technologies have optimized and extended data processing pipelines to maximally extract and separate the weak underlying gastric signals from noise, thereby representing a critical advance over legacy systems.

BSGM as used herein in some embodiments measures the cutaneous dispersion of gastric myoelectrical potentials (typically μV), arising from extracellular ion current flows during depolarization and repolarization of gastric tissues. This encompasses both gastric slow wave activity, generated and propagated by interstitial cells of Cajal (ICC), and coupled smooth muscle contractions. The underlying sources are complex, because multiple waves (typically 3-4) simultaneously propagate through the human stomach, traveling at a slow velocity of ˜3 mm s−1 prior to the terminal antral acceleration. In some embodiments, these features correspond to a scenario where gastric potentials recorded at the body surface cannot be definitively related to a single specific wave sequence, as in electrocardiography, but instead must be considered as a summation of such sources.

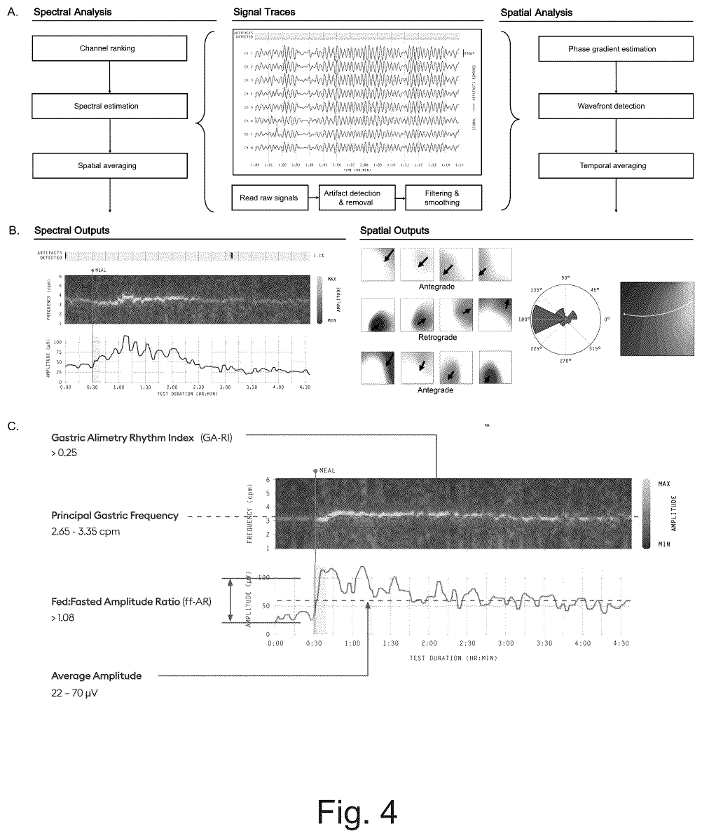

Embodiments can use an EGG morphology that provides a distinct 3 cycle per minute (cpm) waveform, this owing to scenarios when gastric slow waves are entrained to a single frequency, that dominant frequency can be utilitarianly captured in the body-surface potential (FIG. 4).

In addition to gastric activity data, it is utilitarian in some embodiments to measure gastric activity in response to a meal stimuli. In order to achieve the highest quality data and/or data for comparison purposes, testing can be implemented through a standardized system as much as possible.

Embodiments include implementing a Gastric Alimetry test protocol that has the participant fast for at least and/or equal 6 hours and avoid medications modifying gastric function as well as caffeine and nicotine on the day of testing. Embodiments can include fasting for at least 2, 3, 4, 5, 6, 7, 8, 9, 10 hours or more or any value or range of values therebetween in 15 minute increments. Tests can be, in some embodiments, conducted in the morning. The fasting can be linked to the onset of testing (e.g., fasting for at least 2 hours would correspond to starting the testing 120 minutes after food was last consumed).

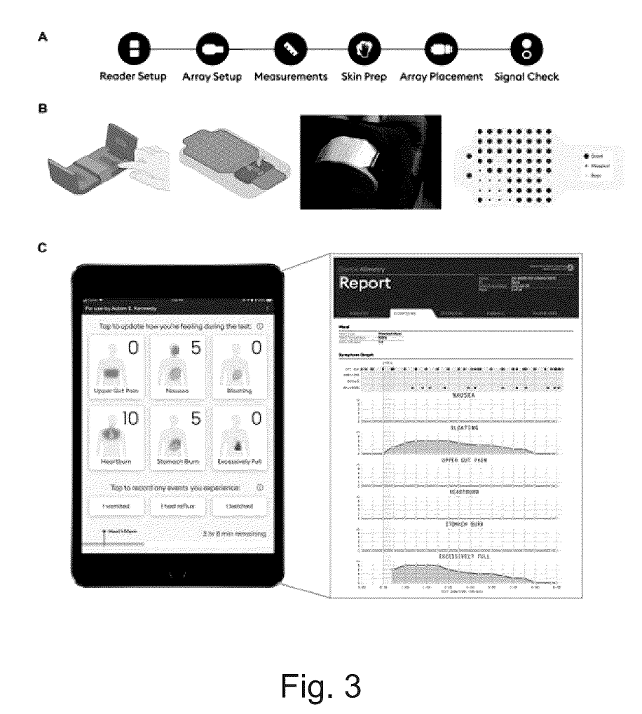

Sensor array placement can be preceded by shaving if utilitarian, followed by skin preparation with an exfoliant conductive gel such as NuPrep® (Weaver & Co, CO, USA) to minimize impedance. The Gastric Alimetry App can be provided with an impedance threshold check prior to allowing recording (see FIG. 3, part A). Fasted recordings are then performed for 30 minutes, by way of example, followed by standardized meal consumed over 10 minutes, and in some embodiments up to a 4-hr postprandial recording. A 4-hr postprandial recording period is utilized in some embodiments to capture a full gastric activity cycle, and because meal responses may be delayed with peak BSGM responses sometimes occurring 2-4 hrs after a meal. However, it has been found that valuable analysis can be performed on data obtained in a testing/recording period of as little as 30 to 60 minutes, and preferably around 45 minutes. Accordingly, in an embodiment, fasted recordings are performed for less than, greater than and/or equal to 10, 15, 20, 25, 30, 35, 40, 45, 50, 55, 60, 65, 70, 75 or more minutes or any value or range of values therebetween in 1 minute increments, contiguous. In some embodiments there is postprandial recording period of less than, greater than and/or equal to 0.2, 0.25, 0.5, 0.75, 1, 1.5, 2, 2.5, 3, 3.5, 4, 4.5, 5, 5.5, 6, 6.5 or 7 hours or more or any value or range of values therebetween in 0.5 minute increments.

### Meal Response

An example of a standard test meal may comprise an off-the-shelf nutrient drink (e.g., Ensure 232 kcal, 250 mL; Abbott Nutrition, IL, USA, available in the United States of America on Oct. 2, 2023, in California, Pennsylvania, Virginia, Delaware, Illinois and/or Texas) and oatmeal energy bar (e.g., a Clif Bar with 250 kcal, 5 g fat, 45 g carbohydrate, 10 g protein, 7 g fiber; Clif Bar & Company, CA, USA, again available in any of those just noted jurisdictions). In embodiments, a different composition can be used. In an embodiment, the calorie consumption of the standard meal is less than, greater than and/or equal to 150, 200, 250, 300, 350, 400 or 450 kcal or any value or range of values therebetween in 10 kcal increments). In an embodiment, this is consumed within less than, greater than and/or equal to 30, 25, 20, 15, 10, 9, 8, 7, 6 or 5 minutes or any value or range of values therebetween in 1 minute increments continuous from beginning to end. In an embodiment, the fat, carbohydrate, protein and/or fiber can be a value that is any one of the just noted numbers that is less than, greater than and/or equal to 30, 35, 40, 45, 50, 55, 60, 65, 70, 75, 80, 85, 90, 95, 100, 125, 150, 175 or 200% or more or any value or range of values therebetween in 1% increments of the just noted numbers (and they values need not be adjusted the same—fat could be lower and carbohydrate could be higher, for example).

To be clear, meals with similar nutritional composition or otherwise utilitarian composition can be substituted per availability or for patients with specific dietary needs, such as those with diabetes or gluten intolerance without substantially/effectively affecting test data. It can be utilitarian to monitor and manage blood sugars in diabetics during testing as hyperglycemia can induce gastric myoelectrical abnormalities. The above meal size is designed to stimulate gastric symptoms in patients with diverse gastric disorders, including milder degrees of functional dyspepsia, and although some patients will find this meal too large, eating only 50% has been reported to adequately profile patients with neuromuscular disorders and centrally-mediated symptoms. In some embodiments, the percent of the just noted meal that is consumed is less than, greater than and/or equal to 25, 30, 35, 40, 45, 50, 55, 60, 65, 70, 75, 80, 85, 90, 95, 100, 125, 150, 175 or 200% or more or any value or range of values therebetween in 1% increments of the noted numbers above, and this is consumed in the aforementioned timeframe.

In an embodiment, nothing is consumed for greater than and/or equal to 0.5, 1, 1.5, 2, 2.5, 3, 3.5, 4, 4.5, 5, 5.5 or 6 hours or more or any value or range of values therebetween in 3 minute increments before and/or after the aforementioned timeframe (and the two need not be the same). In an embodiment, only de minimus foods are consumed (e.g., a mint for example) within those times, while in other embodiments, nothing is consumed.

Embodiments include implementing a standardized BSGM test protocol that includes several steps to optimize recording quality, or at least improve recording quality to a utilitarian level and/or an effective level, such as reducing and/or limiting movement, talking, sleeping (which suppresses gastric activity), and avoiding touching the electrode array to reduce artifact contamination, other than overlying clothes or blankets etc. It is utilitarian in some embodiments that patients are positioned in a comfortable-chair, such as for example that which is reclined at 30, 35, 40, 45, 50, 55, 60, 65, 70 or 75 degrees or any value or range of values therebetween in 1 degree increments, and in some embodiments, with their legs elevated, to reduce and/or avoid abdominal wall contractions. It is also utilitarian that in some embodiments the selected chair can be locked in a set reclined position, or at least prevented from moving more than a certain range (e.g., within 1, 2, 3, 4, 5, 6, 7, 8, 9, 10, 11, 12, 13, 14, 15, 16, 17, 18, 19, 20, 21, 22, 23, 24 or 25 degrees or any value or range of values therebetween in 1 degree increments), so as to reduce a scenario where restless abdominal tensing may contaminate data with electromyographic noise. During the test, patients can mobilize for comfort or bathroom breaks, with for example an on-board accelerometer data being tracked to identify periods of motion (or without such accelerometer data). In an embodiment, the patent is in the chair and/or at least stated/does not move (other than simple arm movements and/or leg movements (cross legs for example) and/or does not change the noted angles for at least 80, 85, 90, 91, 92, 93, 94, 95, 96, 97, 98, 99 or 100% or any value or range of values therebetween in 1% increments for the period of time of monitoring data.

### Symptom Information

Robust symptom profiling concurrent to BSGM testing is utilized in some implementations of the present system, as this can provide clinical utility. Temporal associations between physiological events and symptoms can be used to inform mechanistic interpretations. Accordingly, a patient symptom-logging application (FIG. 3, part C) is provided in at least some embodiments to differentiate symptoms with severity lying on a continuum or specific events.

Alternatively, in some embodiments, this data could be collected manually and later entered into the system for quantification and analysis.

For example, gastrointestinal symptoms including (but not limited to) one or more or all of nausea, bloating, upper gut pain, heartburn, stomach burn, and/or excessive fullness, are assessed on a continuum. In contrast, discrete events such as episodes of vomiting, reflux and/or belching are time stamped, all by way of example.

Continuous symptoms are assessed during the test at suitably granular intervals. For example less than, greater than and/or equal to 2, 3, 4, 5, 6, 7, 8, 9, 10, 11, 12, 13, 14, 15, 16, 17, 18, 19, 20, 21, 22, 23, 24, 25, 26, 27, 28, 29 or 30 or any value or range of values therebetween in 1 increment minute intervals can be used in some implementations. In an embodiment, 10, 15, 20, 25, 30, 35, 40, 45, 50, 55, 60, 70, 80, 90, 100, 125, 150, 175 or 200 or more or any value or range of values therebetween in one increment assessments are made during the test. In an embodiment, the assessments are spaced apart by any one or more the aft aforementioned time intervals.

In order to make the data logging easy to use for a patient, symptom information can be entered via a pictographic interface (such as on a GUI of a computer or smart phone or smart device, etc.) that aids accurate standardized reporting, for example using a 0-10 visual analog scale (where 0 indicates ‘no symptom’ and 10 indicates the ‘most severe extent’ of a symptom). Other suitable alternatives will be appropriate for obtaining symptom information, with varying degrees of user ease and therefore potentially compliance. By way of example, at speech to text system could be used where the patient describes the experience. This could be time stamped for example. Note also, the patient could be prompted by the computer or smart device, such as audibly and/or in a tactile and/or visual manner, etc.

Patient symptom inputs are used to preferably generate a symptom curve (function) that can be used for data analysis (preferably automated By algorithm) and/or a report that can be standardized (preferably automated By algorithm), that covers the course of the test meal (FIG. 4). It has been found that the data correlates effectively with patients' overall symptom burden and quality of life. These symptom curves can be used for numerical analysis and thresholding (preferably in software), while the reports may offer utilitarian clinical aid in combination with BSGM physiological data, as discussed further below (‘BSGM Phenotypes’).

### BSGM—Body Surface Gastric Mapping

BSGM analytics are used, in some embodiments, to generate one or both categories of metrics:
i) spectral metrics which encompass frequency, amplitude, rhythm stability, and meal responses;
ii) spatial metrics which describe spatiotemporal dynamics of slow waves projected to the body surface.

An overview of how these metrics can be derived is provided in FIG. 4 by way of example. In this example, spectral metrics are derived from the spectrogram, akin to a high resolution EGG for example, generated from those channels with the highest SNR on the array (in an embodiment, it is the highest 50, 55, 60, 65, 70, 75, 80, 85, 86, 87, 88, 89, 90, 91, 92, 93, 94, 95, 96, 97, 98 or 99% or any value or range of values therebetween in 1% increments). Spatial metrics can reflect summated depolarizations across the array which can resolve the predominant direction of wave propagation, and the stability of slow wave patterns. In an exemplary embodiment, the signals from the electrode arrays are sufficiently temporally fine that the signals can be utilized to identify wave propagation in the patient. In an exemplary embodiment, features associated with the wave propagation are correlated with the symptom indication information from the patient to identify one or more of the phenotype sets disclosed herein. In an exemplary embodiment, a trained neural network is utilized to evaluate the spatial metrics, and, in conjunction with the input from the patient, or otherwise in conjunction with data based on the input from the patient, any one or the analysis and/or determining actions, etc., detailed herein can be executed. Aspects of a trained neural network will be described in greater detail below. But briefly, in an exemplary embodiment, the neural network can be trained on a sufficient number of spatial metrics and other data sets to enable the product of the trained neural network to make determinations based thereon.

### BSGM Spectral Metrics

Customized BSGM spectral metrics have been developed with reference intervals.

Some exemplary metrics are:
Principal Gastric Frequency
BMI-Adjusted Amplitude
Gastric Alimetry Rhythm Index™ (GA-RI)
Fed:Fasted Amplitude Ratio (ff-AR)

These metrics present a considerable difference from comparable classical EGG metrics to resolve pitfalls, optimize performance, and can provide that one or more or all of the metrics measures a distinct physiological aspect of gastric function. Some considerations in the development of these metrics are summarized as follows, by way of example only and not by way of limitation:
In some embodiments, frequency measurements are susceptible to contamination by high-amplitude low-frequency transients arising from motion artifacts and by periodic adjacent colonic activity, 7 which impair, significantly in some instances) accuracy in ˜5% of cases (refer 9 for further detail). The Principal Gastric Frequency metric can be used to overcome this pitfall that is present in some scenarios/implementations by measuring only the sustained frequency (or weighting the sustained frequency higher than others, such as by way of example, at least 2, 3, 4, 5, 6, 7, 8, 9, 10, 15, 20, 25 or 30 or more times or any value or range of values therebetween in 1 increment) within the plausible gastric range, while excluding other transients and irregularities irrespective of their influence on the power spectrum (or weighting those downward, such as by the inverse of any of those weightings).
Amplitude measurements can be confounded by BMI due to signal attenuation through abdominal adipose. A BMI-adjusted amplitude metric can be used in some embodiments using a multiplicative regression model, enabling comparison of amplitudes across populations up to a current test threshold of BMI 35.9. It has been found that the gastric amplitude metric (including BMI adjustment or not) is particularly useful for differentiating patients, alone or in combination with other metrics as described.
Legacy EGG metrics for assessing the stability of gastric rhythm can include ‘percentage bradygastria’ and ‘percentage tachygastria’, which conflate frequency with rhythm stability, and the ‘instability coefficient’, which is often incorrectly motivated due to its dependence on frequency. A metric of stability can be implemented in the Gastric Alimetry System, GA-RI, which is a measure of concentrated gastric activity within a narrow gastric frequency band over time relative to the residual spectrum. The GA-RI is scaled between 0 (no rhythm stability) and 1 (maximum rhythm stability), and is independent of frequency.
The gastric meal response, as measured by postprandial amplitude curves, demonstrates considerable temporal variability. A study of 110 control subjects showed that the median time of peak amplitude was 1.6 h (IQR 0.7-2.7 h) after meal completion. EGG power-ratio calculations were typically based on shorter intervals, such as the initial 45-minute postprandial period, potentially underestimating the gastric meal response. The revised ff-AR metric measures the gastric response based on the maximum amplitude in any single 1-hour period of a 4-hour postprandial window and is therefore adaptive to variable meal response profiles.

Some embodiments can include arrangements that address one or more or all of the above-noted considerations associated with developing the metrics. In an exemplary embodiment one or more of the above-noted issues are not present or otherwise do not occur or otherwise are remediated for by implementing one or of the teachings detailed herein.

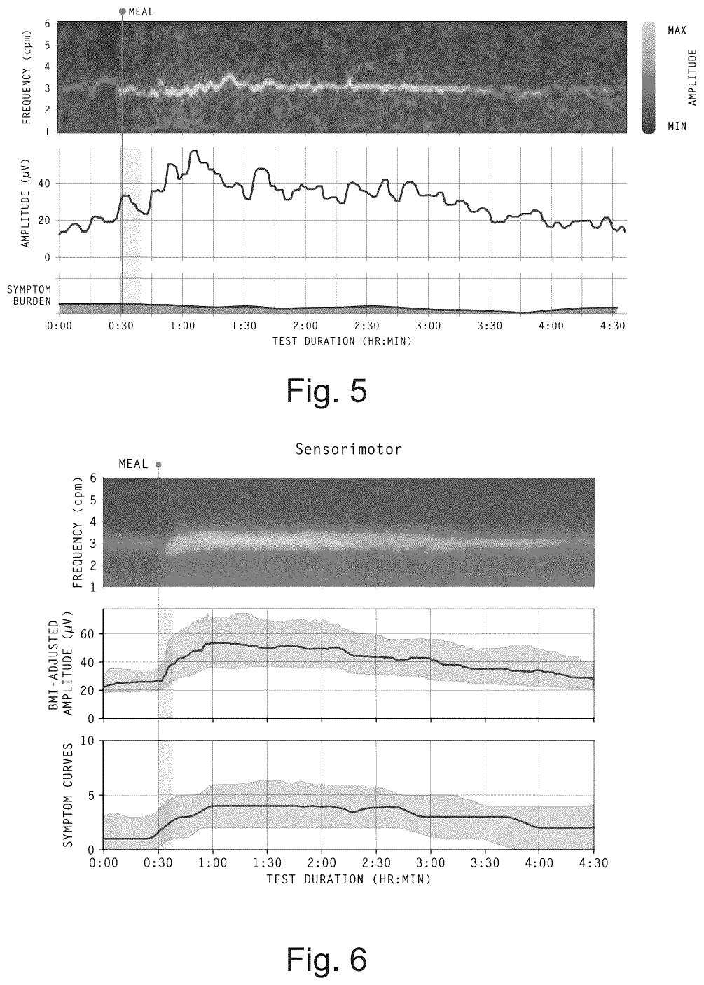

FIG. 5 presents an example output plot for a typical (normal) patient, showing a spectral plot (top), a plot of amplitude extracted (normalised) from the raw electrode data (middle), and a plot of overall symptom burden extracted from the symptom logging App (bottom).

BSGM Reference Intervals and their Interpretation

Embodiments have been practiced. The following is presented in terms of an exemplary implementation for purposes of disclosure and is not presented by way of limitation. Any one or more of the below actions and/or features can be excluded from any of the embodiments, providing that the art enables such, unless otherwise noted.

Reference intervals for the four BSGM spectral metrics were developed from a cohort of healthy volunteers of diverse age, sex and ethnicity, with cross-validation analysis demonstrating external validity. These intervals were generated for participants aged ≥18 years with BMI <35 kg/m2, where >50% of the meal is consumed during the test and <50% of the test duration is affected by artifacts. These reference intervals, summarized FIG. 4, part C, are used to guide clinical interpretations of BSGM data.

Several general considerations are taken into consideration in the design and interpretation of clinical reference intervals for BSGM metrics. These include inappropriately assuming metrics fit Gaussian distributions, and as BMI-adjusted amplitude and ff-AR were found to be skewed, these reference intervals were defined using non-parametric methods. Other pitfalls include incorrectly assuming the central 95% reflects physiological normality, ignoring overlapping distributions between controls and patients, ignoring intra-individual variability, and conflating reference intervals with diagnostic tests.

It will be appreciated that many methods of statistically analysing such data are available in order to assess whether a correlation exists (or not) and the strength of any correlation (or not). The present system is not intended to present novel statistical techniques, nor is it intended to be necessarily limited to the statistical techniques disclosed herein as examples.

The person skilled in the art will be aware of various mathematical techniques for assessing characteristics of a data stream, to provide a numerical measure of a correlation or characteristic.

Similarly, the various thresholds and/or predetermined correlation thresholds applied in order to differentiate and classify data is not intended to be limiting, but rather be examples of what has been found to work effectively.

Further still it will be appreciated that the process of normalising data, is a known technique(s) and potentially necessary to compare data streams. The process may involve one or more separate steps as required. For example, data may be normalised to synchronize with the time a standard meal was ingested etc. Similarly, data may be normalised by applying an offset to allow ‘like-with-like’ comparison, as is a known technique. Further still, normalising may involve combining data from multiple channels (e.g. from multiple electrodes of array 100) into a single curve (function) representative of the gastric activity during the test period (or at least a portion of). Further still, normalising may include discarding data anomalies, such as dropping electrodes with low signal, or anomalies introduced by patient movement etc.

Further still normalizing may involve the minimum value of the function (for example gastric amplitude) being subtracted from the whole function, and/or the function (for example the gastric amplitude) being divided by its sum.

In some embodiments, these described pitfalls are avoided, or at least the chances of such being present are reduced in an effective manner, or at least the occurrence of such is reduced by 70, 80, 90, 95% or more or any value or range of values therebetween in 1% increments, over a statistically significant number of implementations, or over 50, 75, 100, 150, 200, 250 or 300 or more or any value or range of values therebetween in 1 increment implementations relative to that which would be the case, all other things being equal, in the BSGM framework by treating the reference intervals as guides for patient phenotyping, to be viewed holistically alongside patient history and symptom profiles. The effect of demographic parameters (age, sex, and ethnicity) on BSGM have also been evaluated, and while minor differences were found with regard to sex, these differences were sufficiently trivial to allow a single common set of adult reference intervals.

Two-sided reference intervals (Principal Gastric Frequency and BMI-adjusted amplitude) may be reported as 5th and 95th percentiles, whereas one-sided reference intervals (GA-RI and ff-AR) may be reported using the 5th percentile. Five percent of healthy volunteers can therefore be expected to fall outside of the end of an interval.

It is also noted that BSGM is a diagnostic aid, and while the use of reference intervals provides a valuable indication of pathology, it should be integrated with symptom evaluations, clinical history, examination, and other investigations to form a clinical diagnosis.

It is similarly noted that the BSGM metrics represent averages over a test duration, and manual review of spectral plots may still be required to detect transient deviations that may be associated with symptoms but otherwise missed in averaged data tables.

The present teachings include embodiments that quantify and classify a specific set of patient symptom profiles, and their relationships to simultaneously recorded gastric activity. The teachings herein can facilitate quantitative analyses of the role of symptoms in clinical assessment of gastroduodenal disorders at scale. Robust metrics are included to quantify physiological characteristics and symptom profiles into objective symptom phenotypes. Various characteristics, the associated metrics and phenotypes, and their clinical implications are discussed below.

A standardised digital classification framework has been provided that is capable of separating patients into those with:
a) abnormal spectral analyses (suspected neuromuscular pathologies);
b) normal spectral analyses with symptoms correlated to gastric amplitude (sensorimotor, post-gastric, and activity-relieved); and
c) symptoms independent of gastric amplitude (continuous, meal-relieved, meal-induced).

### Continuous Phenotype—Symptom Persistence (No Clinical Correlation Between Symptom Severity and Gastric Activity)

### Gut-Brain Axis Disorder

We have found that having a spectral abnormality is strongly associated with daily symptom severity and poorer quality of life. However, it has been found that for patients having spectral analyses that are normal, and symptom patterns that were independent of gastric amplitude (such as continuous, meal-relieved, and meal-induced patterns) were more strongly correlated with depression and anxiety.

For example, in a test population of patients, we found that between 15-20% remained undifferentiated such that with a normal spectrogram, their symptom curves did not meet the criteria for a predefined pattern (e.g. predefined phenotypes as identified above).

Specifically, patients with a normal spectrogram, considered to indicate an intact gastric neuromuscular apparatus, and a symptom profile unrelated to gastric activity (e.g., continuous, meal-induced, and meal-relieved) have been found to have the strongest correlations with depression and state anxiety scores. Conversely, those with abnormal spectrograms had relatively low depression scores. The important clinical implication, of this finding is that patients can be divided into primarily ‘disorders of gut brain interaction’ (DGBI) vs neuromuscular subgroups. This is expected to allow improved patient selection for principally psychological therapies vs gastric-targeted therapies such as prokinetics and neuromodulation. However, it is also acknowledged that the relationship between symptoms and psychological factors is bidirectional and further work is ongoing to differentiate causal chains of symptom genesis.

For patients with chronic nausea and vomiting disorders but with normal Gastric Alimetry spectral analyses tend to have worse anxiety and/or depression than patients whose symptoms may be explained by gastric neuromuscular abnormalities. Further, of patients with normal BSGM spectral analyses, pre-meal high symptom severity and persistence of high symptoms throughout the test is a phenotype that was highly associated with anxiety and/or depression.

These results indicate that a high premeal symptom severity that persists through the test, may be suggestive of disorders linked to the gut-brain axis. This is typically observed with the symptoms that are high throughout the test and yet do not correlate with the gastric amplitude (See FIG. 8). For example to quantify the symptom persistence, we can define the range metric as the difference between the 95th and 5th percentile of severity for a particular symptom throughout the test. The continuous phenotype is then identified for example by thresholding the range (e.g. range <3) and the 5^{th} percentile of the severity (e.g. 5th percentile >2).

Accordingly, a method may involve monitoring a patient's gastric activity over a test period by obtaining data based on spectral gastric activity with an electrode array concurrently with patient symptom information (for a predetermined set of symptoms) as described above. The degree of correlation between patient symptom information and gastric activity amplitude is assessed with a statistical technique and treatment follows:
(i) treating for a gut-brain axis disorder if the measure indicates a correlation is absent, optionally by not satisfying a predetermined correlation threshold; or
(ii) treating for gastric dysfunction if the measure of said correlation indicates a correlation exists, optionally by satisfying a predetermined correlation threshold.

The patient may be optionally classified as ‘Continuous’ phenotype.

### Sensorimotor Phenotype—Symptom-Gastric Amplitude Clinical Correlation

It has been identified that a subset of patients exhibit symptoms that are tightly time-synchronized with the gastric amplitude, indicating that these symptoms may have a sensorimotor component and may be suggestive of disorders linked to visceral Hypersensitivity (see FIG. 6).

In order to quantify this relationship, we define the symptom amplitude clinical correlation as the correlation coefficient between the symptom severity curve (see for example lowest graph in FIG. 5-9) and gastric amplitude curve (see for example middle graph in FIG. 5-9). The symptom amplitude clinical correlation is thresholded to identify the sensorimotor (e.g. correlation>0.5) phenotype. In an embodiment, where the correlation coefficient is calculated for one or more or all symptom severity curve if a standard deviation is above a predetermined deviation threshold, that predetermined deviation threshold can be 0.05, 0.1, 0.15, 0.2, 0.25, 0.3, 0.35, 0.4, 0.45, 0.5, 0.55, 0.6, 0.65, 0.7, 0.75, 0.8 or more or any value or range of values therebetween in 0.01 increments.

Embodiments include implementing a temporal correlation coefficient as a basis for an indication that certain things are present. By way of example only and not by way of limitation, a maximum temporal correlation coefficient can be used to determine a phenotype for temporal associations between normalized gastric amplitude and normalized symptom severity. In an exemplary embodiment, a sensory motor phenotype is indicated when a maximum temporal correlation coefficient is greater than 0.5.

Accordingly, a method may involve monitoring a patient's gastric activity over a test period by obtaining data based on spectral gastric activity with an electrode array concurrently with patient symptom information (for a predetermined set of symptoms) as described above. Determining a degree of temporal association between the gastric amplitude and continuous symptom severity function(s). The symptoms may be selected from a predetermined set of symptoms and/or for an average symptom function for two or more symptoms selected from the predetermined set of symptoms. If a significant degree of temporal correlation is found, treatment may follow that appropriate for visceral hypersensitivity, for example.

The patient may be optionally classified as ‘Sensorimotor’ phenotype. In particular, a maximum temporal correlation coefficient may used to determine a phenotype for temporal associations between normalised gastric amplitude and normalised symptom severity.

The method may also include calculating a temporal correlation coefficient (for example Pearson's r), and based on the coefficient, assessing the temporal synchronization of the normalised gastric activity amplitude function and a normalised symptom severity function (curve).

Further, the temporal correlation coefficient may be calculated for each symptom severity curve (or an average symptom curve) if a standard deviation is above a predetermined standard deviation threshold.

The temporal correlation coefficient (for example Pearson's r) may be calculated for time lags of ranging from approximately −10 to +10 minutes, with approximately 1 minute steps, and the correlation may for example be taken as the maximum of these values.

### Activity-Relieved and Post-Gastric Phenotypes—Symptom-Amplitude Time Lag.

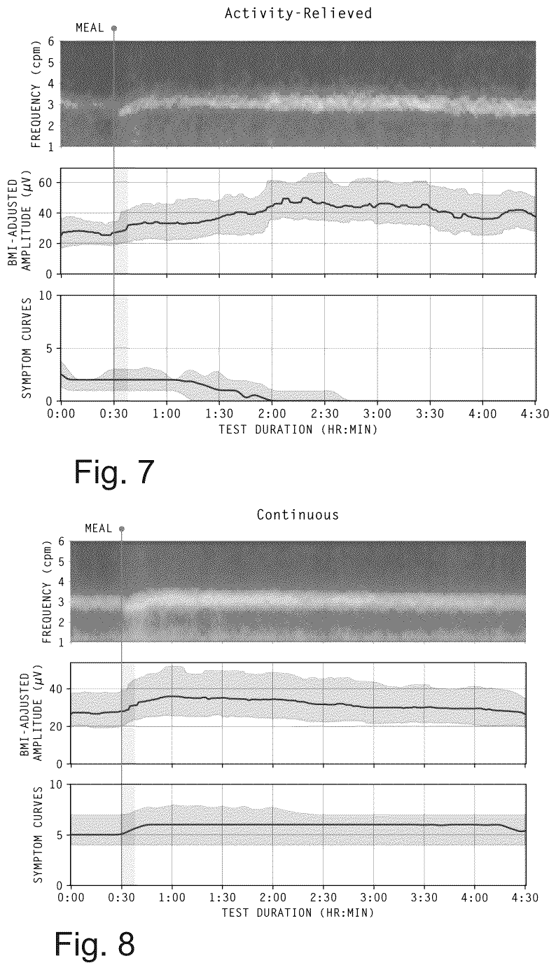

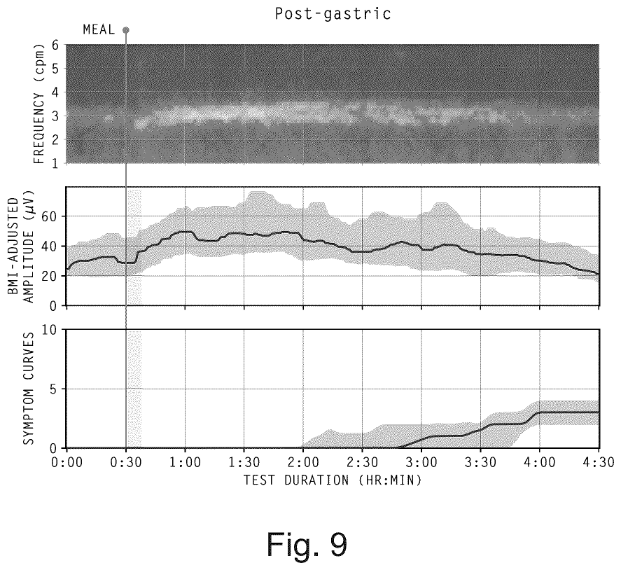

Patients may exhibit symptoms that occur either before the onset or after the conclusion of a physiological gastric meal response, suggesting that symptoms may be related to delayed onset of gastric mixing or a pathology distal to the stomach, respectively (see FIG. 7 & FIG. 9).

As a measure of the extent to which either of these patterns occur, we define the symptom amplitude time lag as the average difference between the cumulative distribution functions of symptom and amplitude (−1 indicates all symptoms occurring before all gastric activity, and +1 all symptoms occurring after gastric activity).

The symptom amplitude time lag is thresholded to identify the activity-relieved (e.g. lag<−0.25, see FIG. 7) or post-gastric (e.g. >0.25, see FIG. 9) phenotypes.

Based on the above scheme, the symptom metrics for the symptom severity curves profiled for nausea, bloating, upper gut pain, heartburn, and stomach burn were computed in Gastric Alimetry tests performed on patients with chronic gastroduodenal symptoms.

The proposed thresholds were used to phenotype symptom curves, with the possibility for a symptom curve to have zero or multiple associated phenotypes. Symptom curves associated with each phenotype were visualized using the median curve and the associated interquartile range. For phenotypes relating symptom severity to gastric amplitude, the median (IQR) amplitude curves and average spectrograms for the patients with one or more symptom matching the phenotype are also shown.

FIGS. 7 and 9 illustrate that the proposed phenotypes produce distinct characterizations of the symptom severity curves and their relationships to time-synchronized gastric activity, and show their relative frequency of occurrence in a large cohort.

Accordingly, a method may involve monitoring a patient's gastric activity over a test period by obtaining data based on spectral gastric activity with an electrode array concurrently with patient symptom information (for a predetermined set of symptoms) as described above. Further the method involves identifying a time lag between the gastric amplitude and one or more symptom severity functions, using statistical techniques, for example cumulative distribution functions (CDFs).

For example an average difference between cumulative distribution functions (CDFs) may be used to assess the time lag between normalised gastric amplitude and a normalised continuous symptom severity function.

Further, a correlation coefficient may be calculated for one or more respective symptom severity curves (or an average of two or more symptom curves). Optionally, a correlation coefficient may be calculated if a standard deviation is above a predetermined deviation threshold.

For example the predetermined deviation threshold may be approximately 0.5 for individual symptom curves, or for example may be 0.1 for an average of two or more symptom curves.

The time lag is quantified as the average difference between the CDF of the normalised gastric amplitude function and the CDF of the normalised symptom severity function.

Accordingly, the time lag is thresholded to determine phenotypes associated with symptoms that either precede or follow gastric activity according to:
a. Post-Gastric phenotype of symptoms following gastric activity is indicated if said time lag is greater than 0.25, or
b. Activity-Alleviated phenotype if symptoms preceding gastric activity is indicated when said time lag is less than −0.25.

Further, the determined phenotypes above may be considered by a clinician to provide targeted therapies to patients according to:
a) The Post-Gastric phenotype may be treated as having small bowel/biliary causes,
b) The Activity-Alleviated phenotype may be treated using a neuromodulator such as mirtazapine,

### Further Embodiments

Embodiments provide relationship(s) of symptom severity curves with concurrent myoelectrical activity of the stomach. Embodiments provide a standardized approach to quantifying and classifying symptom profiles for relating continuous time-of-test symptoms to simultaneously recorded gastric activity. While the proposed approach was developed using the Gastric Alimetry system, it is applicable for any diagnostic test of gastroduodenal function applied in the context of a meal.

In an exemplary embodiment, there is method, including the method action of obtaining data based on measured spectral gastric activity measured with a plurality of electrodes in signal communication with the electrical impulses of a patient. In an exemplary embodiment, the electrodes are part of an electrode array as noted above. In an exemplary embodiment, the measured spectral gastric activity measured with the electrodes as measured during a first temporal test period. In an exemplary embodiment, this method action, the action of obtaining data based on measured spectral gastric activity, can be executed by receiving a data package with data that is directly or indirectly based on the measurements utilizing the plurality of electrodes.

Accordingly, in an exemplary embodiment, this can be executed by a server that is remote from where the measurements are actually being taken. In this regard, the data can be provided to a remote location from the clinic where the measurements are being taken, where the receipt by the server would constitute the action of obtaining. In an exemplary embodiment, the data can be massaged data that is rectified to remove extraneous data channels for example, with the data can be weighted, etc. In an exemplary embodiment, the data package can be received in real time during the action of monitoring, or can be received after completion of measuring, such as one or two or three or more days after the action of measuring. In an exemplary embodiment, the action of obtaining occurs no longer than 5, 10, 15, 20, 25, 30, 35, 40, 50, 60, 90, 120, 180, 250, 300, 350, 400, 500, 600, 700, 800, 900, 1000, 1500, 2000, 2500 or 3000 or any value or range of values therebetween in one increment seconds and/or minutes after the completion of the first temporal test. In an embodiment, some of the data can be acquired well before the end of the first temporal period, such as when the data is being obtained in real time with the measurements. Accordingly, in an exemplary embodiment, the obtained data includes data elements that correspond to data based on measurements at specific sometimes during the first temporal period and the data elements can be obtained within any of the just noted time frames, and in fact can be obtained in shorter time periods, such as, for example, within 4, 3, 2, 1, 0.75, 0.5, 0.25 or 0.1 seconds of the measurements upon which the data elements are based being taken.

Of course, in some embodiments, the action of obtaining the data can be executed by actually taking the measurements utilizing the patch by way of example detailed herein. In an exemplary embodiment, the method actions disclosed herein are executed by a clinician and/or a physician or the combination of the two working under the same authority who is physically present with the patient, who are utilizing a computer or a device adapted to implement one or more the teachings detailed herein, or otherwise have access to a device or a computer system, etc., or otherwise a system, whether directly or via a link, such as the Internet or the like, etc., which device etc. is configured to implement at least one of the actions detailed herein.

In an exemplary embodiment, the method includes the action of obtaining data based on patient symptom information for a predetermined set of symptoms. In this exemplary embodiment, the patient symptom information was or is obtained during at least a portion of the first temporal test. In an exemplary embodiment, as noted above, this can entail having the patient provide output (or input, depending on the perspective) indicating the given sensation that he or she is feeling associated with a given symptom. This output can be scaled data as noted above.

As with the case of the action of obtaining the data based on the measured spectral gastric activity, in this exemplary embodiment, the action of obtaining data based on patient symptom information can also be executed by, for example, obtaining a data package that is based directly or indirectly on the information obtained from the patient. Thus, this action can also be executed remote from the location where the patient is located. Still, in an embodiment, the data can be obtained by the same actor that is “managing” the patient. In an exemplary embodiment, the patient is in a clinic and a clinician who manages the patient (e.g., positions the patient in a given chair for example with a specific posture that is desired for example or provides the general set up for the patient, gives the general instruction for example to the patient, places the electrode patch on the patient, etc.) is co-located with the patient. In an exemplary embodiment, it is the person who manages the patient that obtains the data, and/or it is a local computer that obtains the data, such as for example a computer that has an input device configured to receive output from the recipient, such as by way of example only and not by way limitation, some of the input output systems detailed herein.

In an exemplary embodiment, one or more the method actions detailed herein are executed in a manner where, for example, the patient is located less than greater than and/or equal to 5, 10, 15, 20, 25, 30, 35, 40, 50, 60, 70, 80, 90, 100, 1250, 1500, 2000, 2500, 3000, 3500, 4000, 4500, 5000 or any value or range of values therebetween in one increment feet or miles from the actor executing one or more of the method actions detailed herein, such as the action of obtaining the data detailed herein.

It is briefly noted that any of the features detailed above with respect to the action of obtaining the measure of spectral gastric activity can also correspond to the action of obtaining the data based on the patient symptom of information.

To be clear, in an exemplary embodiment, any one or more of the features detailed herein associated with any one or more feature of any one or more other embodiment corresponds to a disclosure of utilizing those one or more features with one or more features of another embodiment, providing that the art enable such, unless otherwise noted, all in the interest of textual economy. That is, embodiments include any feature disclosed herein utilized in combination with any other feature disclosed herein. Corollary to this is that embodiments include any one or more features disclosed herein that is/are specifically excluded from utilization with any one or other features disclosed herein, providing that the art enable such, unless otherwise noted, in such will not be expanded upon in the interests of textual economy.

In an exemplary embodiment, the method includes the action of determining data indicative of gastric activity amplitude from the measured gastric activity data. In an exemplary embodiment, this can correspond to In an exemplary embodiment, the data indicative of gastric activity amplitude is normalized gastric activity amplitude. In an exemplary embodiment, the action of normalizing is executed by the actor who is determining the data indicative of the gastric activity amplitude. In an exemplary embodiment, this can be implemented according to any of the teachings detailed herein. In an exemplary embodiment, this can be executed utilizing a computer that is configured with software to execute this action in an automated or semiautomated manner.

In an exemplary embodiment, the method also includes the action of correlating the patient symptom information with the data indicative of normalized gastric activity amplitude over the test period. In an exemplary embodiment, the correlations can correspond to those detailed above by way of example only and not by way limitation. An exemplary embodiment, this can be executed utilizing a computer program that is located on a computer, which computer program automatically takes the data detailed above and automatically correlates the data. This computer can also execute the action of evaluating the correlation. In an embodiment, this can be by determining a measure of the correlation.

In an embodiment, the correlation is executed for at least and/or equal to and/or no more than 25, 30, 35, 40, 45, 50, 55, 60, 65, 70, 75, 80, 85, 90, 95 or 100% or any value or range of values therebetween in 1% increments of the total test period. In an exemplary embodiment, correlation is contiguous. In an exemplary embodiment, separate parts of the test period are correlated. By way of example only and not by way of limitation, if movement or some other factor renders some of the data deviant or otherwise reduces the utility of the data, that data might be excluded from the correlation.

That said, any one or more these actions can be executed by a clinician and/or a physician.

The method also includes the identification of a treatment for a gut-brain axis disorder if the evaluation of the correlation indicates no clinical correlation exists. By way of example only and not by way of limitation, a clinical correlation can be based on predetermined thresholds. If the correlation that is determined falls outside a predetermined correlation threshold, in an exemplary embodiment, the method includes identifying the treatment for the gut-brain axis disorder based on such occurrence. Conversely, if the measure of the correlation falls within a predetermined correlation threshold, and identification of a treatment for gastric dysfunction can be executed based on such.

While the above embodiment(s) have often focused on executing the method where the actor need not be one of the parties obtaining the measurements, in an alternate embodiment, the actor obtaining the measurements does not do one or more of the actions, but instead receives results of the actions and acts based thereon. In an exemplary embodiment, there is a method where the actor obtains first data based on measured spectral gastric activity measured with an electrode array during a first temporal test period. Here, the actor actually attaches the electrodes to the patient, and takes the recordings (the actor could be a clinic for example, where a technician or a nurse or a physician is working therefore). This method can further include the action of obtaining second data based on patient symptom information for a predetermined set of symptoms, wherein the patient symptom information was obtained during at least a portion of the first temporal test period. Again, this could be the clinician or the technician receiving the output from the patient. In an exemplary embodiment, the clinician can do the data logging of the symptoms the patient is experiencing, while in other embodiments, the clinician or the clinic is operating a machine that receives the output from the patient, such as by way of example, from an application that the patient is utilizing. Indeed, in an exemplary embodiment, the clinician is in another room away from the patient. Here, the clinician is receiving electronic communication from the patient inputted into a computer co-located with the patient for example. Any device system and/or method or arrangement that can enable the above noted method actions can be utilized in at least some exemplary embodiments.

In an exemplary embodiment, this method further includes the action of providing the first and second data. In an exemplary embodiment, this data is provided into a computer that is linked to a remote server by way of example, which remote server receives the first and second data. This method action can be executed by the clinician coordinating data transfer from the system utilized to detect the electrical signals in the patient and/or the clinician coordinating data transfer from the system utilized to collect the symptoms experienced by the patient. This can be done by placing the hardware utilized to collect the data into signal communication, directly or indirectly, with a remote server by way of example.

In an exemplary embodiment, the method includes the action of receiving third data, and prescribing a treatment based on the third data. This can be any one or more the treatments detailed above. In an exemplary embodiment, instead of or in addition to prescribing a treatment, based on the action of receiving third data, a diagnosis is made about the medical condition afflicting the patient. In an exemplary embodiment, the third data is an evaluation of a correlation of the first data with data indicative of normalized gastric activity amplitude from the second data.

It is briefly noted that embodiments can include the utilization of a product of a trained neural network to execute one or more of the actions detailed herein. By way of example only and not by way limitation, the action of correlating and/or the action of evaluating the correlation can be executed by a product of the trained neural network. In an exemplary embodiment, this is a chip that results from training of the neural network. In an embodiment, the various method actions herein are executed a sufficient number of times to establish a baseline training for the neural network. Upon the training of the neural network, the product thereof is utilized to execute the action of evaluating where the action of correlating or even the action of determining the measure of normalized gastric activity, etc. Any disclosure herein of any analysis and/or determining and/or measuring action corresponds to an alternate disclosure of utilizing executing such with a trained neural network or more accurately, the product of a trained neural work, providing that the art enables such unless otherwise noted.

One utilitarian result of the teachings herein is the introduction of a formal metrics for quantifying the relationship between symptoms and simultaneously recorded gastric activity. In some embodiments, this approach can prove useful for disentangling the complex interconnections that exist between patients' experienced symptoms, gastroduodenal dysfunction, and/or disorders relating to the gut-brain axis.

The proposed phenotypes can be linked to a physiological mechanism, enabling these phenotypes to guide further studies attempting to link symptom phenotypes with long-term outcomes to treatments and interventions. Also, embodiments establish a standardized and fully quantitative system for characterizing Symptoms.

Standardized time-of-test symptom profiles offer a novel approach to classifying patients based on proposed mechanisms of disease. These groupings correlated with chronic symptoms, quality of life, and psychological factors, can now be evaluated for informing mechanism-driven targeted therapies.

Where in the foregoing description reference has been made to elements or integers having known equivalents, then such equivalents are included as if they were individually set forth.

It will of course be realised that while the foregoing description has been given by way of illustrative example(s) of the invention, all such modifications and variations thereto as would be apparent to a person skilled info the art are deemed to fall within the broad scope and ambit of the various aspects of invention as is hereinbefore described and/or defined in the claims.

1.-47. (canceled)

48. A method for monitoring gastric activity with an electrode array patch disposed over a skin surface of a patient to determine a gastrointestinal phenotype, the method comprising:
measuring spectral gastric activity data of the patient with the electrode array over a predetermined test period;
concurrently receiving patient symptom information for a predetermined set of symptoms over the predetermined test period;
determining a gastric activity amplitude over at least a portion of the test period from the measured gastric activity data; and
correlating the patient symptom information with the gastric activity amplitude;
determining a measure of correlation; and
determining a gastrointestinal phenotype based at least in part on the measure of correlation.

49. The method of claim 48, wherein the gastrointestinal phenotype comprises a sensorimotor phenotype.

50. The method of claim 49, wherein the sensorimotor phenotype is associated with postprandial distress syndrome therapies.

51. The method of claim 49, wherein the measure of correlation is indicative of substantial synchronization between a severity of patient symptom information and the gastric activity amplitude.

52. The method of claim 48, wherein the gastrointestinal phenotype comprises a post-gastric phenotype.

53. The method of claim 52, wherein the post-gastric phenotype is associated with small bowel or biliary therapies.

54. The method of claim 52, wherein the measure of correlation is indicative of an increase in severity of patient symptom information following a decrease in the gastric activity amplitude.

55. The method of claim 48, wherein the gastrointestinal phenotype comprises an activity-alleviated phenotype.

56. The method of claim 55, wherein the activity-alleviated phenotype is associated with neuromodulation therapies.

57. The method of claim 55, wherein the measure of correlation is indicative of a decrease in severity of patient symptom information preceding an increase in the gastric activity amplitude.

58. The method of claim 48, wherein the gastrointestinal phenotype comprises a continuous phenotype.

59. The method of claim 58, wherein the continuous phenotype is associated with gut-brain disorder or epigastric pain syndrome therapies.

60. The method of claim 58, wherein a severity of patient symptom information is consistent throughout the predetermined test period and the measure of correlation is indicative of a lack of synchronization between the symptom severity and the gastric activity amplitude.

61. The method of claim 48, further comprising providing the patient with a standardized meal at a predetermined time during the predetermined test period.

62. The method of claim 48, wherein measuring gastric activity data with an electrode array further includes generating spatial information associated with gastric activity of the patient.

63. The method of claim 48, wherein the patient symptom information is received predetermined intervals during the test period.

64. The method of claim 48, wherein the patient symptom information includes a symptom severity metric.

65. The method as claimed in claim 48, wherein the predetermined set of patient symptoms comprise:
a) nausea
b) bloating
c) upper gut pain
d) heartburn
e) stomach burn, or
f) excessive fullness.

66. The method as claimed in claim 48, further comprising determining a measure of temporal association and determining the gastrointestinal phenotype of the patient based on the measure of correlation and the measure of temporal association.

67. A system for monitoring gastric activity of a patient to determine a gastrointestinal phenotype, the system comprising:
an electrode array patch having a plurality of electrodes configured to measure spectral gastric activity data of the patient over a predetermined test period; and
a processor configured to:
receive the measured spectral gastric activity data;
concurrently receive patient symptom information for a predetermined set of symptoms over the predetermined test period;
determine a gastric activity amplitude over at least a portion of the test period from the measured gastric activity data; and
correlate the patient symptom information with the gastric activity amplitude;
determine a measure of correlation;
determine a gastrointestinal phenotype based at least in part on the measure of correlation; and
generate a report comprising at least the determined gastrointestinal phenotype.

68. The system of claim 67, further comprising a connector device coupled to the electrode array patch.

69. The system of claim 67, further comprising a patient logging device for patient symptom information input.

70. The system of claim 67, further comprising a display for displaying the generated report in a digital medium.

71. The system of claim 67, wherein the report comprises a physical medium.

72. The system of claim 67, wherein the gastrointestinal phenotype comprises a sensorimotor phenotype.

73. The system of claim 72, wherein the sensorimotor phenotype is associated with postprandial distress syndrome therapies.

74. The system of claim 72, wherein the measure of correlation is indicative of substantial synchronization between a severity of patient symptom information and the gastric activity amplitude.

75. The system of claim 67, wherein the gastrointestinal phenotype comprises a post-gastric phenotype.

76. The system of claim 75, wherein the post-gastric phenotype is associated with small bowel or biliary therapies.

77. The system of claim 75, wherein the measure of correlation is indicative of an increase in severity of patient symptom information following a decrease in the gastric activity amplitude.

78. The system of claim 67, wherein the gastrointestinal phenotype comprises an activity-alleviated phenotype.

79. The system of claim 78, wherein the activity-alleviated phenotype is associated with neuromodulation therapies.

80. The system of claim 78, wherein the measure of correlation is indicative of a decrease in severity of patient symptom information preceding an increase in the gastric activity amplitude.

81. The system of claim 67, wherein the gastrointestinal phenotype comprises a continuous phenotype.

82. The system of claim 81, wherein the continuous phenotype is associated with gut-brain disorder or epigastric pain syndrome therapies.

83. The system of claim 81, wherein a severity of patient symptom information is consistent throughout the predetermined test period and the measure of correlation is indicative of a lack of synchronization between the symptom severity and the gastric activity amplitude.

84. The system of claim 67, wherein the spectral gastric activity data further includes spatial information associated with gastric activity of the patient.

85. The system of claim 67, wherein the patient symptom information is received at predetermined intervals during the test period.

86. The system of claim 67, wherein the patient symptom information includes a symptom severity metric.

87. The system of claim 67, wherein the predetermined set of patient symptoms comprise:
a) nausea
b) bloating
c) upper gut pain
d) heartburn
e) stomach burn, or
f) excessive fullness.

88. The system of claim 67, wherein the processor is further configured to determine a measure of temporal association.

89. The system of claim 88, wherein the processor is further configured to determine the gastrointestinal phenotype of the patient based on the measure of correlation and the measure of temporal association.
